(19) United States
(12) Patent Application Publication
(10) Pub. No.: US 2023/0233156 A1
(43) Pub. Date: Jul. 27, 2023

(54) BLOOD ABNORMALITY PREDICTION DEVICE, BLOOD ABNORMALITY PREDICTION METHOD, AND PROGRAM

(71) Applicants: RIKEN, Wako-shi, Saitama (JP); AT CO., LTD., Osaka-shi, Osaka (JP)

(72) Inventors: Kei MIZUNO, Wako-shi, Saitama (JP); Yasuyoshi WATANABE, Wako-shi, Saitama (JP); Dan TAKENO, Osaka-shi, Osaka (JP); Takao MACHITANI, Ibaraki-shi, Osaka (JP)

(73) Assignees: RIKEN, Wako-shi, Saitama (JP); AT CO., LTD., Osaka-shi, Osaka (JP)

(21) Appl. No.: 18/003,578

(22) PCT Filed: Dec. 6, 2021
(86) PCT No.: PCT/JP2021/044594

§ 371 Date: Dec. 28, 2022

(30) Foreign Application Priority Data
Dec. 21, 2020 (JP) 2020-211888
Jun. 11, 2021 (JP) 2021-097982

(51) Int. Cl. A61B 5/00; A61B 5/02; A61B 5/107

(52) U.S. Cl. CPC A61B 5/7275; A61B 5/02007; A61B 5/1075; A61B 5/1079; A61B 2576/02

(57) ABSTRACT

There is a need for a technique to determine a presence or absence of morbidity of a lifestyle-related disease and a possibility of future morbidity (risk of morbidity) in a non-invasive manner for a subject. The present disclosure provides a blood abnormality prediction device including, a prediction unit configured to predict a presence or absence of a blood abnormality in a subject on the basis of the information of the image that captures a crown portion of a capillary, wherein the prediction unit is configured to measure one or more selected from the group consisting of an entire width, an apex width, a loop diameter, a venous limb width, and an arterial limb width of the crown portion of the capillary on the basis of the information of the image, to predict the presence or absence of the blood abnormality in the subject from a result of the measurement.

### TECHNICAL FIELD

The present disclosure relates to a blood abnormality prediction device and a blood abnormality prediction method for predicting a presence or absence of a blood abnormality in a subject from a result of measurement on a crown portion of a capillary, and a program for executing the method in a computer.

### BACKGROUND ART

In recent years, methods for determining health status of a subject by imaging capillaries in a fingertip are gaining attention. Such methods include, for example, a blood vessel observation method for determining a presence or absence of subpapillary plexus running along a direction crossing the longitudinal direction of a finger (see Patent document 1), a capillary image processing method for clearly visualizing the morphology of capillaries from a captured image of the capillaries to calculate a length, a thickness, and the number of the capillaries running along the longitudinal direction (see Patent document 2), and a health status evaluation support system for automatically recognizing the shapes of capillaries to calculate the degree of abnormality in the capillaries (see Patent document 3).

### CITATION LIST

### Patent Document

[Patent Document 1] Japanese Patent No. 6186663

[Patent Document 2] Japanese Patent No. 6551729

[Patent Document 3] Japanese Laid-Open Patent Publication No. 2019-106202

### SUMMARY OF INVENTION

### Technical Problem

There is a need for a technique to determine a presence or absence of morbidity of a lifestyle-related disease and a possibility of future morbidity (risk of morbidity) in a subject, in a non-invasive manner for the subject. As described above, several techniques to determine health status of a subject by imaging capillaries have been disclosed. In this respect, it was not identified in these prior documents how the presence or absence of morbidity of a lifestyle-related disease and the possibility of future morbidity in the subject are determined based on the captured images of capillaries. Accordingly, there has been a need for a technique that can rapidly and accurately determine the health status of the subject from the captured images of capillaries in the subject.

To this end, the present disclosure provides a device, a method, and a program for evaluating a presence or absence of morbidity of a lifestyle-related disease represented by vascular abnormalities in a subject, and a risk of future morbidity thereof, from captured images in which at least shapes of crown portions of capillaries are imaged.

### Solution to Problem

The present disclosure is directed to a blood abnormality prediction device including, at least, an image accepting unit configured to accept information of an image that captures a crown portion of a capillary; and a prediction unit configured to predict a presence or absence of a blood abnormality in a subject on the basis of the information of the image, wherein the prediction unit is configured to measure a shape of, specifically one or more selected from the group consisting of an entire width, an apex width, a loop diameter, a venous limb width, and an arterial limb width of the crown portion of the capillary on the basis of the information of the image to predict a presence or absence of a lipid abnormality in the blood of the subject from a result of the measurement. The subject according to the present disclosure may be a healthy subject.

In another aspect of the present disclosure, the prediction unit is configured to compare the result of the measurement with a predetermined threshold value to thereby predict the presence or absence of the blood abnormality in the subject.

Further, in another aspect of the present disclosure, the lipid abnormality in the blood is one or more selected from the group consisting of a low HDL cholesterol level, a high LDL cholesterol level, and a high neutral fat level. Further, the number of examination items determined as being abnormal from among the examination items consisting of an HDL cholesterol level, an LDL cholesterol level, and a neutral fat level in the subject is predicted.

In another aspect of the present disclosure, the prediction unit is configured to measure the arterial limb width and the venous limb width of the crown portion of the capillary on the basis of the information of the image to compare a measured value obtained from a result of the measurement with a predetermined threshold value to thereby predict the presence of the lipid abnormality in the blood when the measured value exceeds the threshold value, the measured value being a measured value of either the arterial limb width or the venous limb width, whichever is greater.

Further, in another aspect of the present disclosure, the prediction unit is configured to measure the arterial limb width and the venous limb width of the crown portion of the capillary on the basis of the information of the image to compare a measured value obtained from a result of the measurement with a predetermined threshold value to thereby predict an examination item determined as being abnormal from among the examination items consisting of an HDL cholesterol level, an LDL cholesterol level, and a neutral fat level in the subject, the measured value being a measured value of either the arterial limb width or the venous limb width, whichever is greater. When the measured value exceeds the threshold value, the number of the items determined as being abnormal is predicted to be greater, and specifically, the number of the items is one, two, or three.

Another aspect of the present disclosure is directed to the blood abnormality prediction device in which the prediction unit measures the apex width of the crown portion of the capillary on the basis of the information of the image to predict the presence or absence of the lipid abnormality in the blood of the subject from a result of the measurement. The lipid abnormality in the blood may be a high LDL cholesterol level.

The present disclosure further provides a blood abnormality prediction method including an image accepting step of accepting information of an image that captures a crown portion of a capillary; and a prediction step of predicting a presence or absence of a blood abnormality in a subject on the basis of the information of the image, wherein the prediction step includes measuring one or more selected from the group consisting of an entire width, an apex width, a loop diameter, a venous limb width, and an arterial limb width of the crown portion of the capillary on the basis of the information of the image to predict a presence or absence of a lipid abnormality in the blood of the subject from a result of the measurement, as well as a program for executing the blood abnormality prediction method in a computer.

### Advantageous Effects of Invention

The present disclosure enables rapid and accurate prediction for a presence or absence of morbidity of a lifestyle-related disease and a risk of future morbidity thereof in a subject in a non-invasive manner. The present disclosure can contribute to early detection and prevention of a lifestyle-related disease as well as to reduced medical expenses since allowing determination of possible risks not only in a subject suffering from the lifestyle-related disease, but in a healthy subject.

### BRIEF DESCRIPTION OF DRAWINGS

FIG. 1 is a schematic view showing a shape of a crown portion of a capillary measured in the present disclosure.

FIG. 2 is a view showing measuring locations in the shape of the crown portion of the capillary measured in the present disclosure.

FIG. 3 is a system flowchart according to Examples of the present disclosure.

FIG. 4 is a plot showing one example of blood abnormality prediction according to Examples of the present disclosure.

FIG. 5 is a graph showing one example of blood abnormality prediction according to Examples of the present disclosure.

FIG. 6 is a plot showing one example of blood abnormality prediction according to Examples of the present disclosure.

FIG. 7 is a plot showing one example of blood abnormality prediction according to Examples of the present disclosure.

### DESCRIPTION OF EMBODIMENTS

A blood abnormality prediction device and a blood abnormality prediction method and program according to the present disclosure are described below in detail on the basis of the description of embodiments.

The blood abnormality prediction device according to the present disclosure includes at least an image accepting unit and a prediction unit. The image accepting unit is configured to accept information of an image that captures a crown portion of a capillary in a subject. The prediction unit is configured to measure a shape of the crown portion of the capillary on the basis of the information of the image accepted by the image accepting unit to predict a presence or absence of a blood abnormality in the subject from a result of the measurement.

The capillary that is an object to be measured in the present disclosure includes not only a subcutaneous blood vessel but a submucosal blood vessel and the like. In the present disclosure, a captured image of capillaries in a fingernail bed is preferably used. This is because a technique already developed by the inventors allows acquisition of a clear captured image of the capillaries using a non-invasive method imposing a reduced burden on the subject.

In the present disclosure, an imaging unit for capturing an image of the crown portion of the capillary may be included. For example, an image captured by using methods described in the past patent publications by the inventors is preferably used as the captured image used in the present disclosure. Specifically, the methods described in Patent documents 1 and 2 as indicated above are used. That is, the information of the captured image of the capillaries in the fingernail bed can be obtained by an imaging means equipped with an imaging device, such as CCD, CMOS, which images a nail bed portion of the subject's finger illuminated by, for example, visible light or infrared light. The information of the captured image is preferably subject to image-processing to clearly visualize the shapes of the capillaries.

The crown portion of the capillary is also called a loop (capillary loop). The crown portion of the capillary is a site connecting from an artery to a vein and formed directly under the skin. In the case of the capillaries in the fingernail bed, the crown portion of the capillary refers to a hairpin-shaped tip portion formed by a bend in a blood vessel running in the longitudinal direction of the finger.

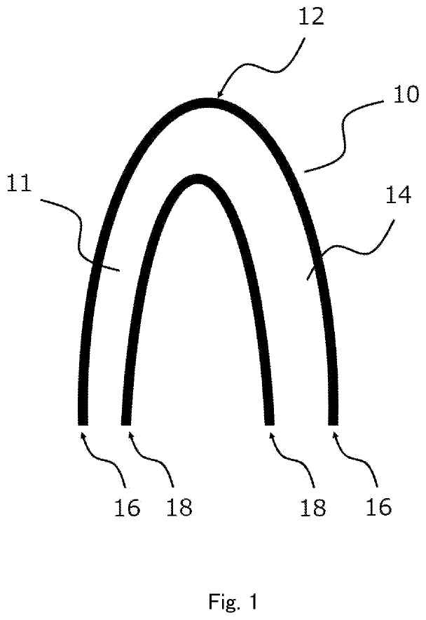

FIG. 1 shows a schematic view of the crown portion of the capillary. The crown portion 10 of the capillary is composed of an arterial limb 11, an apex 12, and a venous limb 14. A blood vessel on the artery side is called the arterial limb 11 and a blood vessel on the vein side is called the venous limb 14, with the apex 12 of the crown portion being a transitioning portion.

Further, in a planar image (two-dimensional image) in which the crown portion of the capillary in the fingernail bed is imaged, a blood vessel wall observed on the outer side of the loop is herein called a loop outer wall 16 and a blood vessel wall observed on the inner side of the loop is called a loop inner wall 18.

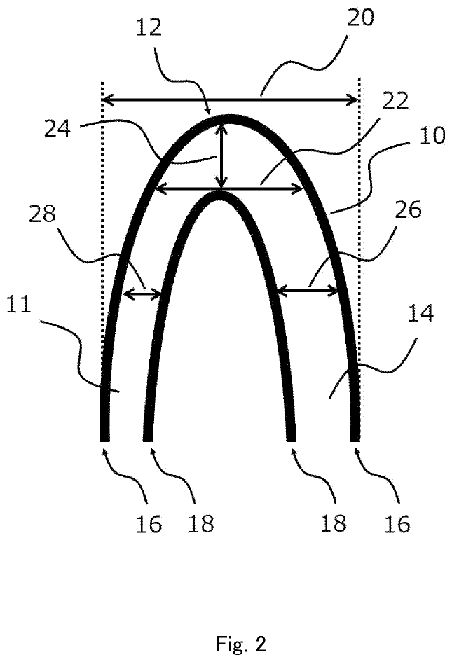

The prediction unit is configured to measure the shape of the crown portion of the capillary in the captured image, preferably a planar image, of the crown portion of the capillary accepted by the image accepting unit. Specifically, the prediction unit is configured to measure the shape of the crown portion of the capillary, i.e., one or more lengths selected from the group consisting of an entire width, an apex width, a loop diameter, a venous limb width, and an arterial limb width, on the basis of the information of the image. FIG. 2 shows a specific example of measuring locations in the crown portion of the capillary. In FIG. 2, the measuring locations in the shape of the crown portion 10 of the capillary are shown by bidirectional arrows. The measuring locations in the shape of the crown portion 10 of the capillary based on the planar image of the crown portion 10 of the capillary are described below with reference to FIG. 2.

The entire width 20 is a width of the crown portion 10 of the capillary in the transverse direction of the subject’ finger, the crown portion 10 of the capillary including the arterial limb 11 and the venous limb 14, and refers to a length of the width from the loop outer wall 16 of the arterial limb 11 to the loop outer wall 16 of the venous limb 14.

The apex width 22 is a width of a space formed within the blood vessel in the crown portion 10 of the capillary. In FIG. 2, it corresponds to a length of a chord of a circular segment formed by the loop outer wall 16 of the apex portion 12. More preferably, the apex width 22 is a length of a straight line representing a chord of the loop outer wall 16 of the apex portion 12 and being tangent to an apex of the loop inner wall 18.

The loop diameter 24 is a diameter of the blood vessel constituting the apex 12 of the crown portion 10 of the capillary and refers to a length from the loop outer wall 16 of the apex 12 to the loop inner wall 18 of the apex 12.

The venous limb width 26 is a diameter of the venous limb 14 and refers to a length from the loop inner wall 18 to the loop outer wall 16 of the venous limb 14. The arterial limb width 28 is a diameter of the arterial limb 11 and refers to a length from the loop inner wall 18 to the loop outer wall 16 of the arterial limb 11.

Numerical values representing the lengths of the entire width, the apex width, the loop diameter, the venous limb width, and the arterial limb width of the crown portion of the capillary may be determined on the basis of statistics calculated from results of measurement based on the information of the image (for example, mean value, maximum value, minimum value, median value, mode value, etc., from obtained multiple numerical values).

The present disclosure enables prediction of the presence or absence of a blood abnormality in the subject from the measured values representing the shape of the crown portion of the capillary. The subject according to the present disclosure may be a healthy subject. According to the present disclosure, it is possible to identify, among healthy subjects, a healthy subject who does not have a confirmed disease due to the blood abnormality, but has a tendency to the blood abnormality, so that a future or potential morbidity risk in the healthy subjects can be recognized.

The inventors found that a high correlation was obtained between the result of the measurement on the shape of the crown portion of the capillary and quantified information obtained by quantifying the blood abnormality in the subject, and thus arrived at the present disclosure. The quantified information obtained by quantifying the blood abnormality in the subject may be not only one blood test value but a combination of multiple blood test values. In the case of the combination of multiple blood test values, the number of blood test items determined as being abnormal from among the blood test items in comparison to a reference value, a numerical value obtained by scoring each of multiple test values according to severity to combine by, for example, addition or multiplication, or a numerical value obtained by weighting each of multiple blood test values to combine by, for example, addition or multiplication may be used.

The blood abnormality that is an object to be predicted in the present disclosure is specifically lipid abnormalities in the blood, and includes suspicions of such blood abnormalities. In the present disclosure, it is possible to rapidly predict in a non-invasive method whether or not the subject has the lipid abnormalities in the blood.

The lipid abnormality in the blood is one or more selected from the group consisting of a condition of having a measured value of HDL cholesterol in the blood lower than a reference value, a condition of having a measured value of LDL cholesterol higher than a reference value, and a condition of having a measured value of neutral fat higher than a reference value, and is also called hyperlipidemia. It is known that those who have the lipid abnormalities in the blood show a higher risk of developing diseases, such as arteriosclerosis, acute pancreatitis, cerebral infarction, myocardial infarction, renal disorder.

In the present disclosure, it is possible to predict, from the measured values representing the shape of the capillary in the subject, whether or not the blood of the subject has one or more selected from the group consisting of a condition of having a low HDL cholesterol level, a condition of having a high LDL cholesterol level, and a condition of having a high neutral fat level. The conditions of having a high LDL cholesterol level and of having a high neutral fat level mean a condition in which a blood test value obtained from the subject is higher as compared to a predetermined reference value, such as a diagnosis criterion. The condition of having a low HDL cholesterol level means a condition in which a blood test value obtained from the subject is lower as compared to the reference value.

The prediction unit according to the present disclosure is configured to compare the result of the measurement on the shape of the crown portion of the capillary with a predetermined threshold value to thereby predict the presence or absence of the blood abnormality in the subject. The threshold value can be suitably provided by analyzing using statistical methods the results of the measurement on the shapes of the crown portions of the capillaries and the blood test values, which are collected from multiple subjects.

The present disclosure may include a display unit. A specific example of the display unit includes a display. The display unit is configured to display a result predicted by the prediction unit, for example, “blood abnormality suspected” or “no blood abnormality suspected,” to a user.

The present disclosure provides a blood abnormality prediction method including an image accepting step of accepting information of an image that captures a crown portion of a capillary; and a prediction step of predicting a presence or absence of a blood abnormality in a subject on the basis of the information of the image, wherein the prediction step includes measuring one or more selected from the group consisting of an entire width, an apex width, a loop diameter, a venous limb width, and an arterial limb width of the crown portion of the capillary on the basis of the information of the image to predict the presence or absence of the blood abnormality in the subject from a result of the measurement. The present disclosure also provides a program for executing the blood abnormality prediction method in a computer.

### Examples

While the present disclosure will be described in greater detail with reference to Examples, the present disclosure is not limited to the following Examples.

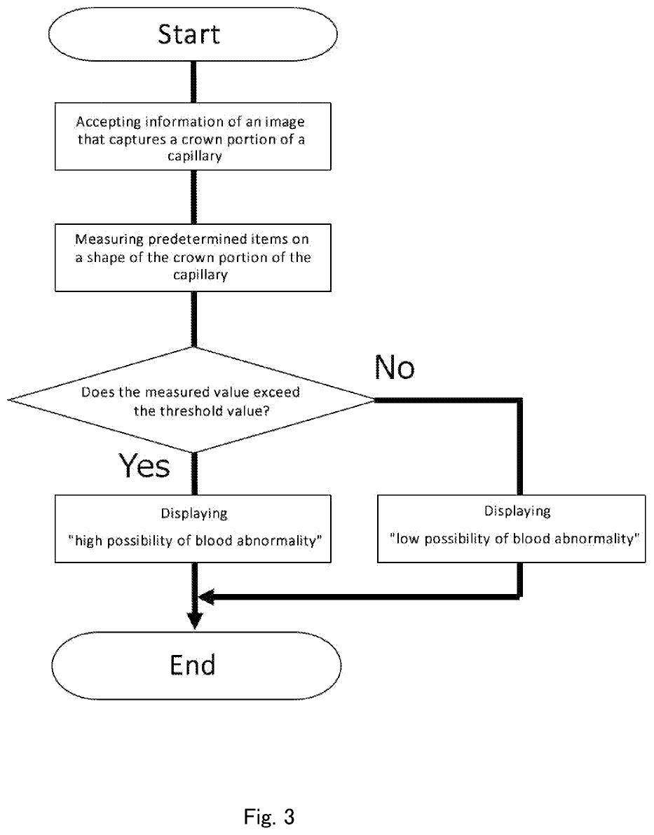

A blood abnormality prediction device in Examples includes an image accepting unit, a prediction unit, and a display unit. FIG. 3 shows a system flowchart for the blood abnormality prediction device in Examples.

In Examples, the image accepting unit accepts information of an image that captures a crown portion of a capillary. Further, the prediction unit measures predetermined items on the crown portion of the capillary on the basis of the information of the image accepted by the image accepting unit to obtain a measured value. The prediction unit further compares the measured value with a threshold value. When the measured value exceeds the threshold value, the display unit displays “high possibility of blood abnormality,” and when the measured value does not exceed the threshold value, the display unit displays “low possibility of blood abnormality.”

Specific examples of blood abnormality prediction in a subject in Examples will be described below.

1. Prediction of the Number of Items Indicating Blood Lipid Abnormalities in a Subject

In this Example, a presence or absence of a blood lipid abnormality, more specifically, the number of blood lipid examination items determined as being abnormal from among the examination items is predicted from measured values of an arterial limb width and a venous limb width at a crown portion of a capillary in a subject. The validity of the prediction in the Example was studied using images of crown portions of capillaries and test values relating to blood lipids in healthy subjects.

Captured images of capillaries in 1020 healthy subjects who consented to this study were used to measure the arterial limb width and the venous limb width. Then, an HDL cholesterol level, an LDL cholesterol level, and a neutral fat level obtained from blood tests in the same subjects were used to divide the subjects into four groups (zero abnormal items, one abnormal item, two abnormal items, and three abnormal items) according to the number of test items determined as being abnormal based on diagnostic criteria (revised in 2012, Japan Atherosclerosis Society). For each group, the measured values of either the arterial limb width or the venous limb width, whichever is greater, were calculated.

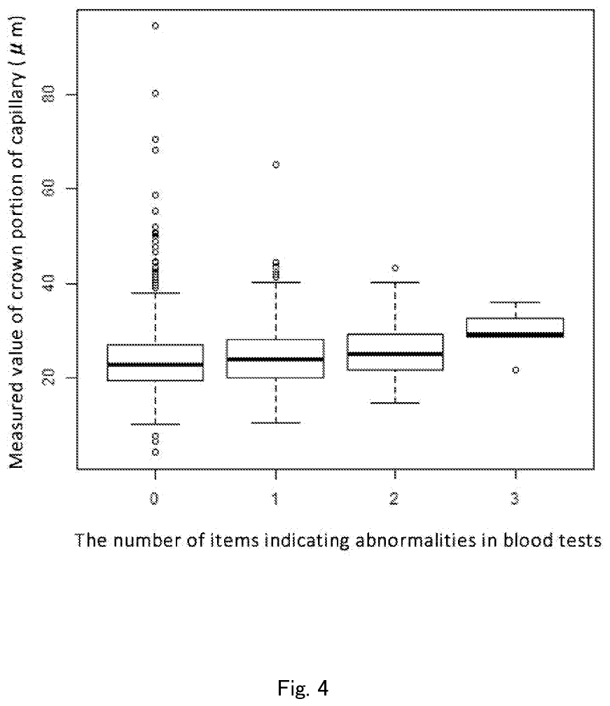

FIG. 4 shows results of analysis for the captured images of capillaries for each of the number of the items indicating abnormalities of blood lipids. While the median values of the measured values obtained from the captured images of the capillaries in the subjects having the zero, one, and two abnormal items were 22.8 μm, 23.9 μm, and 25.0 μm, respectively, the median value from the three abnormal items was 29.0 μm. It was thus found that the number of the items indicating the abnormalities in the blood tended to be greater when the measured value was high.

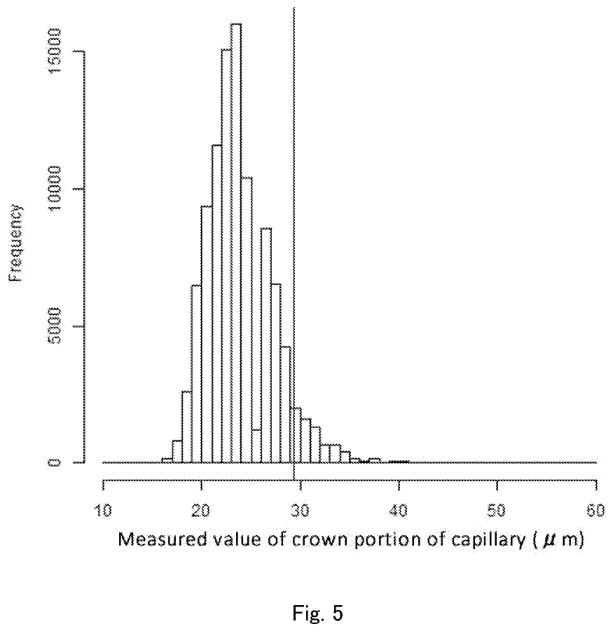

Five samples were randomly extracted from the measured values obtained from the captured images of the capillaries in the subjects having the zero, one, and two abnormal items to calculate the median value thereof. FIG. 5 shows results of 100,000 times calculations. This resulted in 5.5% of the calculated values exceeding 29.0 μm that is the median value from the three abnormal items, and the measured values from the subjects having the two or less items indicating the blood abnormalities were determined to rarely exceed 29.0 μm. Accordingly, it was shown in the Example that whether or not the subject has the three items indicating abnormalities in the blood can be predicted with high probability according to whether or not the measured value from the captured image of the capillary in the subject exceeds the threshold value of 29.0 μm.

2. Prediction of LDL Cholesterol Level in a Subject

In this Example, a presence or absence of an abnormality in an LDL cholesterol level is predicted from a measured value of an apex width at a crown portion of a capillary in a subject. The validity of the prediction in the Example was studied by using images of crown portions of capillaries and LDL cholesterol values in the blood in healthy males between 50 and 59 years old who consented to this study to identify a correlation between the two values.

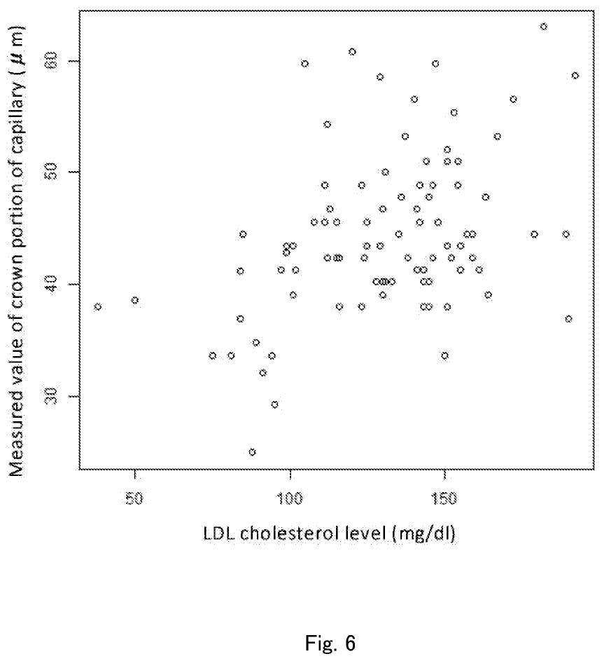

FIG. 6 shows results of the study for the correlation between the LDL cholesterol levels and the measured values for the crown portions of the capillaries from 88 subjects remaining after removing subjects having abnormal values that are a measured value from the image of the capillaries of less than 20 μm and an LDL cholesterol level of 200 mg/dl or more. The calculated correlation coefficient was 0.41 and a high correlation was obtained between the two values.

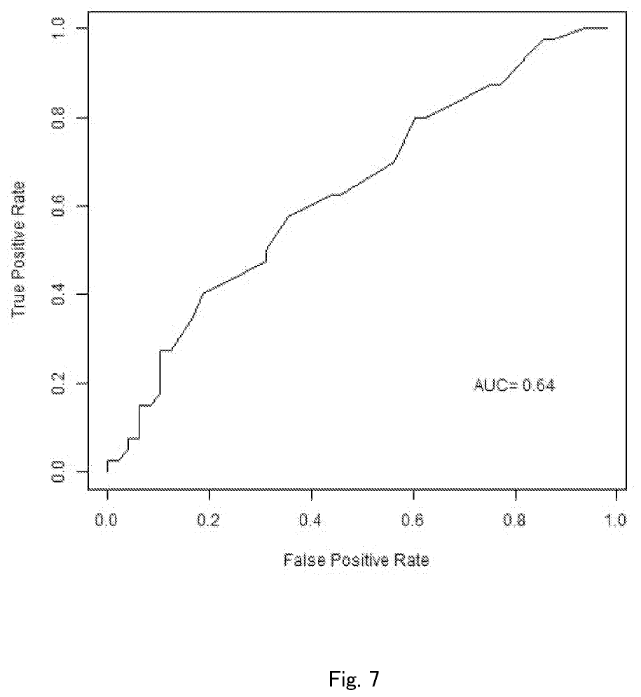

True positive rates and false positive rates for diagnosis based on the measured values from the crown portions of the capillaries are calculated with an LDL cholesterol of 140 mg/dl or more set as being abnormal based on diagnostic criteria (revised in 2012, Japan Atherosclerosis Society), in order to generate an ROC (Receiver Operating Characteristic) curve. FIG. 7 shows the generated ROC curve. The area under the curve (AUC: Area Under the Curve) in the Example was 0.64 and the optimal cutoff value was 47.80 μm. Based on the foregoing results, it is shown that the Example can be sufficiently utilized as a non-invasive primary screening for high LDL cholesterol levels by setting the threshold value for the measured value of the apex width of the crown portion of the capillary to 47.80 μm.

### DESCRIPTION OF REFERENCE CHARACTERS

10 Crown portion of capillary
11 Arterial limb
12 Apex
14 Venous limb
16 Loop outer wall
18 Loop inner wall
20 Entire width
22 Apex width
24 Loop diameter
26 Venous limb width
28 Arterial limb width

1. A blood abnormality prediction device comprising at least:
an image accepting unit configured to accept information of an image that captures a crown portion of a capillary in a subject; and
a prediction unit configured to predict a presence or absence of a blood abnormality in the subject on the basis of the information of the image,
wherein the prediction unit is configured to measure one or more selected from the group consisting of an entire width, an apex width, a loop diameter, a venous limb width, and an arterial limb width of the crown portion of the capillary on the basis of the information of the image to predict a presence or absence of a lipid abnormality in the blood of the subject from a result of the measurement.

2. The blood abnormality prediction device according to claim 1, wherein the prediction unit is configured to measure the arterial limb width and/or the venous limb width of the crown portion of the capillary on the basis of the information of the image to predict the presence or absence of the lipid abnormality in the blood of the subject from a result of the measurement.

3. The blood abnormality prediction device according to claim 1, wherein the prediction unit is configured to measure the arterial limb width and the venous limb width of the crown portion of the capillary on the basis of the information of the image to compare a measured value obtained from a result of the measurement with a predetermined threshold value to thereby predict the presence of the lipid abnormality in the blood when the measured value exceeds the threshold value, the measured value being a measured value of either the venous limb width or the arterial limb width, whichever is greater.

4. The blood abnormality prediction device according to claim 3, wherein the lipid abnormality in the blood is one or more selected from the group consisting of a low HDL cholesterol level, a high LDL cholesterol level, and a high neutral fat level.

5. The blood abnormality prediction device according to claim 1, wherein the prediction unit is configured to measure the arterial limb width and the venous limb width of the crown portion of the capillary on the basis of the information of the image to compare a measured value obtained from a result of the measurement with a predetermined threshold value to thereby predict a number of examination items determined as being abnormal from among the examination items consisting of an HDL cholesterol level, an LDL cholesterol level, and a neutral fat level in the subject, the measured value being a measured value of either the arterial limb width or the venous limb width, whichever is greater.

6. The blood abnormality prediction device according to claim 1, wherein the prediction unit measures the apex width of the crown portion of the capillary on the basis of the information of the image to predict the presence or absence of the lipid abnormality in the blood of the subject from a result of the measurement.

7. The blood abnormality prediction device according to claim 6, wherein the lipid abnormality in the blood is a high LDL cholesterol level.

8. The blood abnormality prediction device according to claim 3, wherein the subject is a healthy subject.

9. A blood abnormality prediction method comprising:
an image accepting step of accepting information of an image that captures a crown portion of a capillary in a subject; and
a prediction step of predicting a presence or absence of a blood abnormality in the subject on the basis of the information of the image,
wherein the prediction unit is configured to measure one or more selected from the group consisting of an entire width, an apex width, a loop diameter, a venous limb width, and an arterial limb width of the crown portion of the capillary on the basis of the information of the image to predict a presence or absence of a lipid abnormality in the blood of the subject from a result of the measurement.

10. A program for executing the blood abnormality prediction method according to claim 9 in a computer.

11. The blood abnormality prediction device according to claim 5, wherein the subject is a healthy subject.

12. The blood abnormality prediction device according to claim 7, wherein the subject is a healthy subject.
